(19) United States
(12) Patent Application Publication
(10) Pub. No.: US 2020/0085668 A1
(43) Pub. Date: Mar. 19, 2020

(54) ELECTRIC WALKING ASSISTIVE DEVICE FOR MULTIMODE WALKING TRAINING AND THE CONTROL METHOD THEREOF

(71) Applicant: NATIONAL YANG-MING UNIVERSITY, TAIPEI CITY (TW)

(72) Inventor: Chung-Huang Yu, TAIPEI CITY (TW)

(21) Appl. No.: 16/479,314

(22) PCT Filed: Jan. 20, 2017
(86) PCT No.: PCT/CN2017/071990

§ 371 Date: Nov. 22, 2019

(51) Int. Cl. A61H 3/04; A63B 23/04

(52) U.S. Cl. CPC A61H 3/04; A61H 2201/0176; A63B 23/04

(57) ABSTRACT

The present invention provides a plurality of training modes for an electric walking rehabilitative device, including: constant speed mode, adaptive speed mode, strengthening push/pull mode, weight bearing mode, high frequency disturbance random speed mode, low frequency perturbation variable speed mode, direction control by handle push/pull forces mode, and slope mode, to improve the user's walking speed, muscle strength, balance, speed adjustment, direction control and slope walking.

### FIELD OF THE INVENTION

The present invention relates to the field of electric walking rehabilitative device, and more specifically, the electric walking rehabilitative device provides a variety of walking training modes.

### BACKGROUND OF THE INVENTION

Electric walking rehabilitative devices are an essential aid for users who still have the ability to walk but rely on support to maintain balance dynamically. However, in addition to providing support, electric walking rehabilitative device also provide various rehabilitation training to enhance the user's walking ability. The improvement of walking ability includes gait cycle, stride frequency, stride length, pace, step position, and muscle strength. Muscle strength is not just about the legs, but also the muscles of the hands, waist, and torso. Directional control training can be further advanced only when muscle strength and coordination meet the criteria. Most importantly, all training modes should be people-oriented and suitable for the user.

Most of the electric walking rehabilitative devices on the market perform a single function, or adapt to a single situation, and therefore cannot be individually adjusted for different users.

### SUMMARY OF THE INVENTION

The present invention relates to an electric rehabilitative device that provides multi-mode walking training mode for users of various conditions and walking abilities.

A walking rehabilitative device for multi-mode walking training, comprises: a mobile platform, which mounts a moving mechanism; a user zone which is a ground adjacent to the mobile platform, wherein the user zone provides a user for standing; a gait sensing module disposed on the mobile platform, wherein the gait sensing module senses the user's feet through non-contact sensing means and outputs a gait characteristic information of the user; a control system disposed on the mobile platform, wherein all modules and the moving mechanism of the walking rehabilitative device are electrically connected to the control system respectively; the control system comprises a trajectory program to control the mobile platform to perform predetermined trajectories, including going straight, left turn and right turn; the control system acquires information and data from all modules of the rehabilitation device, then analyzes and calculates, and controls the rehabilitation device to performs predetermined movement modes.

The walking rehabilitative device further comprises:

a resistance module mounted on the mobile platform and electrically connected to the moving mechanism for adjusting the movement resistance of the walking rehabilitative device;

a multi-axis sensing module, including a plurality of sensors respectively disposed on the left handle and the right handle for sensing the force vector applied by the left and right hands of the user to the left and right handles, and correspondingly output the user's right hand force vector and left hand force vector;

an obstacle sensing module, including a plurality of sensors respectively disposed around the periphery of the mobile platform for sensing whether an obstacle is encountered when the walking rehabilitative device moves, and obtaining the distance between the walking rehabilitative device and the obstacle;

a tilt sensing disposed on the mobile platform for sensing that the mobile platform is tilted forward or rearward;

a stepping force sensing module is embedded in a sole or insole worn by the user, or in a training track, and the control system is electrically connected to the stepping force sensing module.

The invention further includes control methods of the rehabilitative device to perform the following move modes: constant speed mode, adaptive speed mode, high frequency disturbance random speed mode, and low frequency perturbation variable speed mode.

The move modes further include strengthening push/pull mode, affected-foot weight-bearing mode, affected-hand weight-bearing mode, affected-foot-and-hand weight-bearing mode, direction control mode, and slope mode.

The constant speed mode is utilized to train the user to control the walking speed.

The strengthening push/pull mode is utilized to enhance the muscle strength of the user, firstly the feet, followed by the hands, waist and torso.

The weight-bearing mode includes the affected-foot weight-bearing mode, the affected-hand weight-bearing mode, and the affected-foot-and-hand weight-bearing mode. This training is utilized to enhance the muscle strength of the user's affected hands and feet, and train the user to maintain the balance between the affected and healthy side of the hands and feet.

The high frequency disturbance random speed mode is to allow the device to move at a high frequency disturbance random speed to train the user to control his walking speed in the presence of interference.

The low frequency perturbation variable speed mode is to allow the device to move at a low frequency perturbation variable speed to train the user to adjust the walking speed in time.

The direction control mode is to train the user to control the walking direction.

The slope mode is utilized to train the user to control the walking speed and center of gravity when walking uphill and downhill, and to perform a higher-level of muscle strength training.

### BRIEF DESCRIPTION OF THE DRAWINGS

FIG. 1 is a schematic view of the structure of the walking rehabilitative device.

FIG. 2 is a block diagram of the module of the walking rehabilitative device.

### DETAILED DESCRIPTION OF THE INVENTION

For the convenience of the description of the present invention, the central idea expressed in the above summary of the invention is expressed by way of specific examples. Various items in the embodiments are depicted in terms of ratios, dimensions, amounts of deformation, or displacements that are suitable for illustration, and are not drawn to the proportions of actual elements, as set forth above.

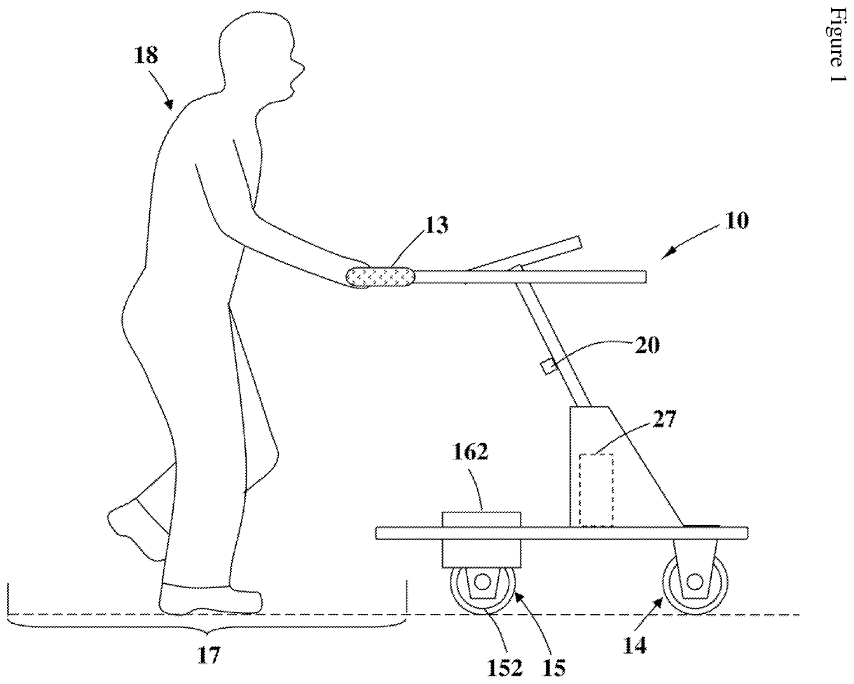

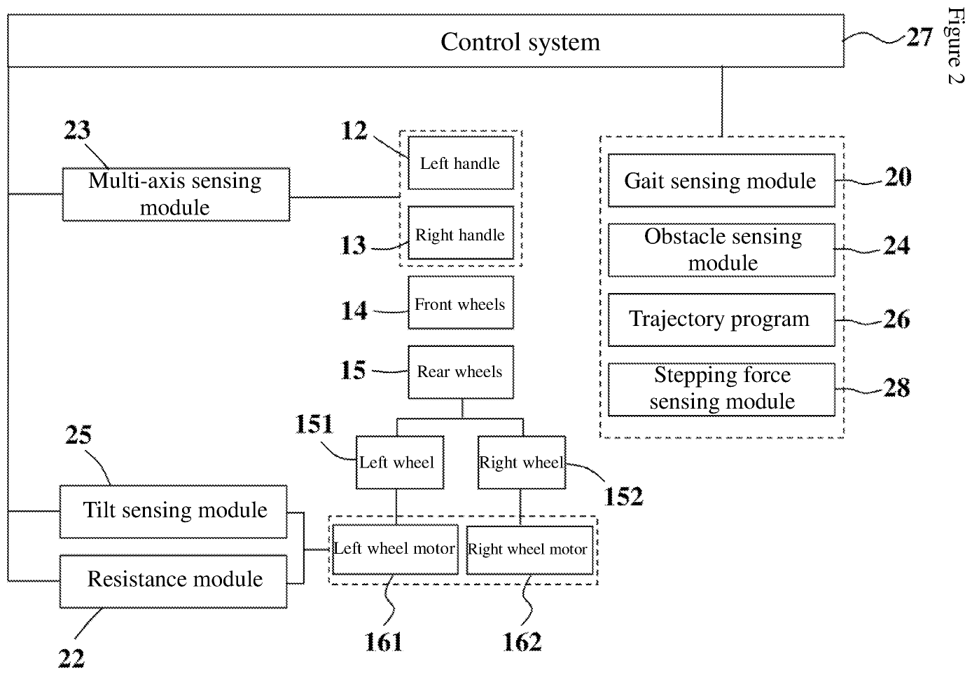

As shown in FIG. 1 and FIG. 2, the walking rehabilitative device of the present invention comprises: a mobile platform 10; a left handle 12 and a right handle 13 connected to the mobile platform 10; a moving mechanism disposed on the mobile platform 10, wherein the moving mechanism is a pair of front wheels 14 and a pair of rear wheels 15; the rear wheel 15 includes a left wheel 151 and a right wheel 152, wherein the left wheel 151 and the right wheel 152 are controlled by a left wheel motor 161 and a right wheel motor 162 respectively; a user zone 17 is a ground area adjacent to the mobile platform 10, a user 18 stands in the user zone 17, and the left and right hands of the user hold the left handle 12 and right handle 13 respectively; the user's elbow bending angle is 25°-35°.

The walking rehabilitative device further comprises a gait sensing module 20, a resistance module 22, a multi-axis sensing module 23, an obstacle sensing module 24, a tilt sensing module 25, and a trajectory program 26 to generate a predetermined trajectory; the above-mentioned modules are electrically connected to a control systems 27 respectively; the control system 27 is further electrically connected to the stepping force sensing module 28 embedded in a sole or insole worn by the user, or in a training track; the control system acquires and analyzes the information and data from all modules of the walking rehabilitative device, and thereby generates the predetermined trajectory.

The gait sensing module senses the user's feet through the non-contact sensing means and outputs a gait characteristic information of the user. The gait characteristic information includes, but is not limited to, a gait cycle, a step frequency, a stride, a pace, a linear distance between a midpoint of the two sole and a specific point of the walking rehabilitative device (hereinafter referred to as a first human-machine distance d1), and whether the user is located in the aforementioned user zone; the non-contact sensing means includes, but is not limited to, an image, a laser, an infrared, an ultrasonic wave, or any sensing module applied to the user's feet.

The resistance module 22 is electrically connected to the left wheel motor 161 and the right wheel motor 162 for increasing the internal resistance of the motors, reducing the motor rotation speed, increasing the motor torque, allowing the user to move the walking rehabilitative device under appropriate resistance.

The multi-axis sensing module 23 is utilized to sense a force vector applied by the left and right hands of the user to the left and right handles, and correspondingly output a user's right hand force vector (VFr) and a left hand force vector (VF1).

The obstacle sensing module 24 is utilized to sense whether an obstacle is encountered when the walking rehabilitative device moves, and obtain a distance between the walking rehabilitative device and the obstacle (D0).

The tilt sensing module 25 is utilized to sense that the mobile platform 10 is tilted forward or rearward. In a embodiment of the present invention, if the mobile platform 10 is tilted backward, the walking rehabilitative device is going uphill; if the mobile platform 10 is tilted forward, the walking rehabilitative device is going downhill.

The trajectory program 26 controls the rotation speeds of the left wheel motor 161 and the right wheel motor 162 to allow the walking rehabilitative device to generate a predetermined trajectory such as a linear movement, a left turn, and a right turn within a predetermined distance.

The walking rehabilitative device of the present invention provides the following training mode through the control system.

The training mode includes: constant speed mode (referred to as M1 mode); adaptive speed mode (referred to as M2 mode); strengthening push/pull mode (referred to as M5 mode); weight bearing mode (referred to as M6 mode, further including M6-1 mode, M6-2 mode, and M6-3 mode); high frequency disturbance random speed mode (referred to as DRS mode); low frequency perturbation variable speed mode (referred to as PVS mode); direction control by handle push/pull forces mode (referred to as PFD mode, further including PFD-1 mode, PFD-2 mode, and PFD-3 mode); slope mode (referred to as SL mode).

The M1 mode is a constant speed mode, the walking rehabilitative device and its control system perform the following control steps, comprising:

Step 1: the control system determines whether the user is located in the user zone through the information from the gait sensing module; if “the user is located in the user zone” is true, proceed to step 2; if false, the walking rehabilitative device does not move or pause the movement, and the control system continues to determine.

Step 2: the control system controls the walking rehabilitative device to move at a predetermined speed (Vs). The predetermined speed (Vs) is system default or set by therapist through the operation interface of the control system.

Step 3: repeat steps 1 and 2 until the end of training, or forced to stop due to an emergency (such as a user falling).

In the M1 mode, the user walks at a predetermined speed of the walking rehabilitative device to improve the walking speed of the user.

The M2 mode is an adaptive speed mode, the walking rehabilitative device and its control system perform the following control steps, comprising:

Step 1: the control system determines whether the user is located in the user zone through the information from the gait sensing module; if “the user is located in the user zone” is true, proceed to step 2; if false, the walking rehabilitative device does not move or pause the movement, and the control system continues to determine.

Step 2: the control system controls the walking rehabilitative device to move at a predetermined speed (Vs). The predetermined speed (Vs) is system default or set by therapist through the operation interface of the control system.

Step 3: the control system acquires the user walking speed from the gait sensing module and calculates the average walking speed (Vn) of the user.

Step 4: the control system compares the predetermined speed (Vs) of the walking rehabilitative device with the average walking speed (Vn) of the user; if {|Vn−Vs|>ϵV} is true, the control system controls the walking rehabilitative device to move at a adaptive speed (Vs1) according to the calculation result of {Vs1=min (Vn, Vu)}; if |Vn−Vs|≤ϵV is true, return to step 2; wherein Vu is set by therapist through the operation interface of the control system; if Vu is not set, Vu is the maximum speed of the walking rehabilitative device, which is preset by the system according to the specification and rotation speed of the motor; (ϵV) is the tolerance value

Step 5: repeat steps 1 to 4 until the end of training, or forced to stop due to an emergency (such as a user falling).

In the M2 mode, the walking rehabilitative device first moves at a predetermined speed (Vs), and then changes to Vs1 according to the user's walking speed.

The M5 mode is a strengthening push/pull mode, the walking rehabilitative device and its control system perform the following control steps, comprising:

Step 1: the control system determines whether the user is located in the user zone through the information from the gait sensing module; if “the user is located in the user zone” is true, proceed to step 2; if false, the walking rehabilitative device does not move or pause the movement, and the control system continues to determine.

Step 2: the control system acquires the right handle reaction force value (|Fr|) and the left handle reaction force value (|Fl|) from the multi-axis sensing module.

Step 3: The control system compares the right handle reaction force value (|Fr|) and the left handle reaction force value (|Fl|) with a right preset threshold (Frth) and a left preset threshold (Flth); if {|Fr|>Frth & |Fl|>Flth} is true, proceed to step 4; if false, return to step 1; wherein the preset threshold (Frth), (Flth) are system default or set by therapist through the operation interface of the control system;

Step 4: the control system acquires the first human-machine distance d1 from the gait sensing module

Step 5: The control system compares the first human-machine distance (d1), the right handle reaction force value (FR), and the left handle reaction force value (FL). If the formula (1) is satisfied, it means the user applies a push force to the walking rehabilitative device. If the formula (2) is satisfied, it means the user applies a pull force to the walking rehabilitative device. Either formula (1) or formula (2) is satisfied, proceed to step 6; if neither formula (1) nor formula (2) is satisfied, return to step 1;

{(d1<dznu) & (Fr<0) & (Fl<0)} formula (1)

{(d1>dznl) & (Fr>0) & (Fl>0)} formula (2)

wherein (dznu) is the upper limit of neutral distance; (dznl) is the lower limit of neutral distance; (dznu) and (dznl) are system default.

Step 6: The walking rehabilitative device moves according to the pushing direction or the pulling direction at a predetermined speed (Vs);

Step 7: repeat steps 1 to 6 until the end of training, or forced to stop due to an emergency (such as a user falling).

In step 6 of the M5 mode, the control system can also activates the resistance module, increase the current of the motor, increase the reverse torque of the motor, and allow the user to move the walking rehabilitative device under appropriate resistance. The user's pushing or pulling force must be increased to move the walking rehabilitative device.

In the M5 mode, the pushing and pulling force exerted by the user on the walking rehabilitative device are used as a judging criterion to start the walking rehabilitative device to move and/or continue to move. Only when the user's leg muscles, waist muscles, trunk muscles, and hand muscles all meet the expected criteria and can coordinate with each other, the walking rehabilitative device can be successfully pushed or pulled.

The M6-1 mode is an affected-foot weight-bearing mode, the walking rehabilitative device and its control system perform the following control steps, comprising:

Step 1: the control system determines whether the user is located in the user zone through the information from the gait sensing module; if “the user is located in the user zone” is true, proceed to step 2; if false, the walking rehabilitative device does not move or pause the movement, and the control system continues to determine.

Step 2: The control system acquires the user's affected side stepping force value (Ta) from the stepping force sensing module.

Step 3: The control system compares the affected side stepping force value (Ta) with a stepping force threshold value (Tth). If {Ta>Tth} is true, proceed to step 4; if false, return to step 1; wherein the threshold value (Tth) is system default or set by therapist through the operation interface of the control system.

Step 4: The control system detects a duration (Time_Ta) of {Ta>Tth} and compares the duration (Time_Ta) of {Ta>Tth} with a default duration (Time_th). If {Time_Ta<Time_th} is true, return to step 1; if {Time_Ta≥Time_th} is true and the control system acquires the user's healthy side stepping force value (Tc) from the stepping force sensing module, the control system controls the walking rehabilitative device to move a predetermined distance at the predetermined speed (Vs). The predetermined distance is user's stride length, system default or set by therapist through the operation interface of the control system.

Step 5: repeat steps 1 to 4 until the end of training, or forced to stop due to an emergency (such as a user falling).

The M6-2 mode is an affected-hand weight-bearing mode, the walking rehabilitative device and its control system perform the following control steps, comprising:

Step 1: the control system determines whether the user is located in the user zone through the information from the gait sensing module; if “the user is located in the user zone” is true, proceed to step 2; if false, the walking rehabilitative device does not move or pause the movement, and the control system continues to determine.

Step 2: the control system acquires an affected-hand force vector (Va) and a healthy-hand force vector (Vc) from the multi-axis sensing module;

Step 3: the control system compares the affected-hand force vector (Va) with affected-hand force vector threshold (Vath) and compares the healthy hand force vector (Vc) with a the healthy hand force vector (Vcth); if {|Va|>Vath & |Vc|>Vcth} is true, proceed to step 4; if false, return to step 1; wherein the preset threshold (Vath), (Vcth) are system default or set by therapist through the operation interface of the control system;

Step 4: the control system controls the walking rehabilitative device to move a predetermined distance at the predetermined speed (Vs). The predetermined distance is user's stride length, system default or set by therapist through the operation interface of the control system.

Step 5: repeat steps 1 to 4 until the end of training, or forced to stop due to an emergency (such as a user falling).

The M6-3 mode is an affected-foot-and-hand weight-bearing mode, the walking rehabilitative device and its control system perform the following control steps, comprising:

Step 1: the control system determines whether the user is located in the user zone through the information from the gait sensing module; if “the user is located in the user zone” is true, proceed to step 2; if false, the walking rehabilitative device does not move or pause the movement, and the control system continues to determine.

Step 2: the control system acquires the user's affected side stepping force value (Ta) from the stepping force sensing module; the control system acquires the affected-hand force vector (Va) and the healthy-hand force vector (Vc) from the multi-axis sensing module.

Step 3: the control system compares the affected side stepping force value (Ta) with a stepping force threshold value (Tth); the control system compares the affected-hand force vector (Va) with affected-hand force vector threshold (Fath) and compares the healthy hand force vector (Vc) with a the healthy hand force vector (Vcth); if {Ta>Tth} and {|Va |>Vath & |Vc |>Vcth} is true, proceed to step 4; if false, return to step 1; wherein the preset threshold (Tth), (Vath), and (Vcth) are system default or set by therapist through the operation interface of the control system.

Step 4: the control system detects a duration (Time_Ta) of {Ta>Tth} and compares the duration (Time_Ta) of {Ta>Tth} with a default duration (Time_th). If {Time_Ta<Time_th} is true, return to step 1; if {Time_Ta≥Time_th} is true and the control system acquires the user's healthy side stepping force value (Tc) from the stepping force sensing module, the control system controls the walking rehabilitative device to move a predetermined distance at the predetermined speed (Vs). The predetermined distance is user's stride length, system default or set by therapist through the operation interface of the control system.

Step 5: repeat steps 1 to 4 until the end of training, or forced to stop due to an emergency (such as a user falling).

The M6-1, M6-2 or M6-3 mode is suitable for users with hand, foot, or one-sided hemiplegia. Such users are often accustomed to using the healthy side as the main support, so that the muscle strength of the affected side is getting weaker. The M6-1 mode trains and enhances the muscle strength of the user's affected foot, and assists the user in controlling and coordinating the support balance of the affected side and the healthy side. The M6-2 mode trains and enhances the muscle strength of the user's affected hand and assists the user in controlling and coordinating the support balance of the affected side and the healthy side. Through the M6-3 mode, the muscle strength of the user's affected hands and feet is trained and improved, and the user is assisted in controlling and coordinating the support balance of the affected and the healthy side hands and feet.

The DRS mode is a high frequency disturbance random speed mode, the walking rehabilitative device and its control system perform the following control steps, comprising:

Step 1: the control system determines whether the user is located in the user zone through the information from the gait sensing module; if “the user is located in the user zone” is true, proceed to step 2; if false, the walking rehabilitative device does not move or pause the movement, and the control system continues to determine.

Step 2: the control system controls the walking rehabilitative device to high-frequency alternately moves at the predetermined speed (Vs) and a random speed (Vrandom); wherein the occurrence and duration of the predetermined speed (Vs) and the random speed (Vrandom) are randomly controlled by the control system, so that the walking rehabilitative device generates high frequency disturbance random speed movement; the predetermined speed (Vs) is constant, and the random speed (Vrandom) is faster or slower than the predetermined speed (Vs); wherein predetermined speed (Vs) is system default or set by therapist through the operation interface of the control system; wherein the random speed (Vrandom) is system default, set by therapist through the operation interface of the control system, or randomly generated by the control system based on the predetermined speed (Vs).

Step 3: repeat steps 1 and 2 until the end of training, or forced to stop due to an emergency (such as a user falling).

In the DRS mode, the walking rehabilitative device that moves at a high frequency disturbance random speed interferes with the user's walking. The users must overcome this disturbance to walk at their own walking speed to train the user effectively control their walking speed under external interference.

The PVS mode is a low frequency perturbation variable speed mode, the walking rehabilitative device and its control system perform the following control steps, comprising:

Step 1: the control system determines whether the user is located in the user zone through the information from the gait sensing module; if “the user is located in the user zone” is true, proceed to step 2; if false, the walking rehabilitative device does not move or pause the movement, and the control system continues to determine.

Step 2: the control system controls the walking rehabilitative device to low-frequency alternately move at the predetermined speed (Vs) and a variable speed (Vv); wherein the occurrence and duration of the predetermined speed (Vs) and the variable speed (Vv) are randomly controlled by the control system, so that the walking rehabilitative device generates low frequency perturbation variable speed movement; the predetermined speed (Vs) is constant, and the variable speed (Vv) is faster or slower than the predetermined speed (Vs); wherein predetermined speed (Vs) is system default or set by therapist through the operation interface of the control system; wherein the variable speed (Vv) is system default, set by therapist through the operation interface of the control system, or randomly generated by the control system based on the predetermined speed (Vs).

Step 3: repeat steps 1 and 2 until the end of training, or forced to stop due to an emergency (such as a user falling).

In the PVS mode, the walking rehabilitative device that moves at a low frequency perturbation variable speed interferes with the user's walking. The users must overcome this disturbance to walk at their own walking speed to train the user effectively control their walking speed under external interference.

The PFD-1 mode is a direction control mode, the walking rehabilitative device and its control system perform the following control steps, comprising:

Step 1: the control system determines whether the user is located in the user zone through the information from the gait sensing module; if “the user is located in the user zone” is true, proceed to step 2; if false, the walking rehabilitative device does not move or pause the movement, and the control system continues to determine.

Step 2: the control system controls the walking rehabilitative device to move with a predetermined trajectory at the predetermined speed (Vs) or the adaptive speed (Vs1); wherein the predetermined trajectory is generated by trajectory program and including linear movement, left-turn, and right-turn.

Step 3: repeat steps 1 and 2 until the end of training, or forced to stop due to an emergency (such as a user falling).

In the PFD-1 mode, the walking rehabilitative device guides the user to move straight, left or right to train the user's direction control.

The PFD-2 mode is a direction control mode, the walking rehabilitative device and its control system perform the following control steps, comprising:

the control system determines whether the user is located in the user zone through the information from the gait sensing module; if “the user is located in the user zone” is true, proceed to step 2; if false, the walking rehabilitative device does not move or pause the movement, and the control system continues to determine.

Step 2: the control system acquires the user's right hand force vector (VFr) and the left hand force vector (VFl) from the multi-axis sensing module

Step 3: the control system compares the user's right hand force vector (VFr) and the left hand force vector (VFl) with a default force vector threshold (VFth); if {(VFl−VFr)>VFth} is true, the control system accelerates the left wheel motor and decelerates the right wheel motor to turn the walking rehabilitative device to the right; if {(VFr−VFl) >VFth} is true, the control system accelerates the right wheel motor and decelerates the left wheel motor to turn the walking rehabilitative device to the left; wherein the default force vector threshold (VFth) is system default or set by therapist through the operation interface of the control system.

Step 4: repeat steps 1 to 3 until the end of training, or forced to stop due to an emergency (such as a user falling).

In the PFD-2 mode, the user controls the moving direction of walking rehabilitative device by the thrust of the left and right hands. In this mode, the therapist can provide a S-shaped training track, a rectangular training track, or a training track with obstacles to train the user to control the walking rehabilitative device to go straight, turn left, turn right, and avoid on the above training track.

The PFD-3 mode is a direction control mode, the walking rehabilitative device and its control system perform the following control steps, comprising:

Step 1: the control system determines whether the user is located in the user zone through the information from the gait sensing module; if “the user is located in the user zone” is true, proceed to step 2; if false, the walking rehabilitative device does not move or pause the movement, and the control system continues to determine.

Step 2: the control system acquires the user's right hand force vector VFr and the left hand force vector VFl from the multi-axis sensing module.

Step 3: the control system compares the user's right hand force vector (VFr) and the left hand force vector (VFl) with a default force vector threshold (VFth); if {(VFl−VFr) >VFth} is true, the control system accelerates the left wheel motor and decelerates the right wheel motor to turn the walking rehabilitative device to the right; if {(VFr−VFl) >VFth} is true, the control system accelerates the right wheel motor and decelerates the left wheel motor to turn the walking rehabilitative device to the left; wherein the default force vector threshold (VFth) is system default or set by therapist through the operation interface of the control system; the control system acquires the distance between the walking rehabilitative device and the obstacle (D0) from obstacle sensing module; the control system compares the distance (Do) with a preset upper threshold (Duth) and a preset lower threshold (Dlth), either {Do >Duth} or {Do<Dlth} is satisfied, the control system controls the walking rehabilitative device to stop and return to step 2, or activate a automatic obstacle avoidance mode or a correction mode to enable the walking rehabilitative device to continue moving and avoid obstacles; or the control system monitors the trajectory of the walking rehabilitative device and compares the trajectory with the predetermined trajectory; If the direction and angle of the movement trajectory deviate significantly from the predetermined trajectory, the control system controls the walking rehabilitative device to stop and return to step 2, or activate a correction mode to correct the direction and angle of the movement to return to the predetermined trajectory.

Step 4: repeat steps 1 to 3 until the end of training, or forced to stop due to an emergency (such as a user falling).

In the PFD-3 mode, the user leads the moving direction of the walking rehabilitative device, but when the walking rehabilitative device is too close to or too far from the obstacle, or is too far away from the predetermined trajectory, the walking rehabilitative device stops moving, allowing the user to correct it; or the walking rehabilitative device automatically bypasses obstacles or automatically corrects the direction to help the user correct the direction of the walking rehabilitative device, so as to train the user's ability to avoid obstacles.

The SL mode is a slope mode, the walking rehabilitative device and its control system perform the following control steps, comprising:

Step 1: the control system determines whether the user is located in the user zone through the information from the gait sensing module; if “the user is located in the user zone” is true, proceed to step 2; if false, the walking rehabilitative device does not move or pause the movement, and the control system continues to determine.

Step 2: the control system senses that the mobile platform is tilted forward or rearward through tilt sensing module. If the mobile platform is tilted backward, the walking rehabilitative device is going uphill, proceed to step 3; if the mobile platform is tilted forward, the walking rehabilitative device is going downhill, proceed to step 4.

Step 3: the control system acquires the right handle reaction force value (|FR|) and the left handle reaction force value (|FL|) from the multi-axis sensing module; the control system compares the right handle reaction force value (|Fr|) with a right preset threshold (Frth) and compares the left handle reaction force value (|Fl|) with a left preset threshold (Flth); wherein the preset threshold (Frth), (Flth) are system default or set by therapist through the operation interface of the control system; if { Fr ≥Fru>0 & Fl ≥Flu >0 } is true, indicating that the user applies a pulling force to the walking rehabilitative device, the control system starts the motor to move the walking rehabilitative device at a predetermined speed (Vs).

Step 4: the control system acquires the right handle reaction force value (|Fr|) and the left handle reaction force value (|Fl|) from the multi-axis sensing module; the control system compares the right handle reaction force value (|Fr|) and the left handle reaction force value (|Fl|) with a right preset threshold (Frl) and a left preset threshold (Fll); wherein the preset threshold (Frl), (Fll) are system default or set by therapist through the operation interface of the control system; if {Fr≤Frl<0 & Fl Fll<0 } is true, indicating that the user applies a pushing force to the walking rehabilitative device, the control system starts the motor to move the walking rehabilitative device at a predetermined speed (Vs).

Step 5: repeat steps 1 to 4 until the end of training, or forced to stop due to an emergency (such as a user falling).

The SL mode is utilized to train the user to control the walking speed and center of gravity when walking uphill and downhill, and to perform a higher-level of muscle strength training.

What is claimed is:

1. A walking rehabilitative device for multimode walking training, comprising:
a mobile platform, which mounts a moving mechanism;
a user zone, which is a ground adjacent to the mobile platform;
wherein the user zone provides a user for standing;
a gait sensing module disposed on the mobile platform;
wherein the gait sensing module senses the user's feet through non-contact sensing means and outputs a gait characteristic information of the user;
a control system disposed on the mobile platform;
wherein all modules and the moving mechanism are electrically connected to the control system respectively;
wherein the control system further comprises a trajectory program to control the mobile platform to perform predetermined trajectories, including going straight, left turn and right turn;
wherein the control system acquires information and data from all modules of the rehabilitation device, then analyzes and calculates, and controls the rehabilitation device to performs predetermined movement modes.

2. The rehabilitative device according to claim 1, further comprising:
a resistance module disposed on the mobile platform and electrically connected to the moving mechanism for adjusting the movement resistance of the rehabilitative device;
a multi-axis sensing module, including a plurality of sensors respectively disposed on a left handle and a right handle for sensing the force vector applied by the left and right hands of the user to the left and right handles, and correspondingly output the user's left-hand force vector and right-hand force vector;
an obstacle sensing module, including a plurality of sensors respectively disposed around the periphery of the mobile platform for sensing whether an obstacle is encountered when the rehabilitative device moves, and obtaining the distance between the rehabilitative device and the obstacle;
a tilt sensing module disposed on the mobile platform for sensing that the mobile platform is tilted forward or rearward;
a stepping force sensing module is embedded in a sole or insole worn by the user, or in a training track, and electrically connected to the control system.

3. A control method for the rehabilitative device of claim 1, wherein the control method enables the rehabilitative device to perform movement modes comprising: constant speed mode, adaptive speed mode, high frequency disturbance random speed mode, and low frequency perturbation variable speed mode.

4. An control method for the rehabilitative device of claim 2, wherein the control method enables the rehabilitative device to perform movement modes comprising: strengthening push/pull mode, affected-foot weight-bearing mode, affected-hand weight-bearing mode, affected-foot-and-hand weight-bearing mode, direction control mode, and slope mode.

5. The control method for the rehabilitative device according to claim 3, wherein the constant speed mode comprising:
Step 1: the control system determines whether the user is located in the user zone through the information from the gait sensing module; if “the user is located in the user zone” is true, proceed to step 2; if false, the walking rehabilitative device does not move or pause the movement, and the control system continues to determine;
Step 2: the control system controls the rehabilitative device to move at a predetermined speed (Vs); the predetermined speed (Vs) is system default or set by therapist through the operation interface of the control system;
Step 3: repeat steps 1 and 2 until the end of training, or forced to stop due to an emergency (such as a user falling).

6. The control method for the rehabilitative device according to claim 3, wherein the adaptive speed mode comprising:
Step 1: the control system determines whether the user is located in the user zone through the information from the gait sensing module; if “the user is located in the user zone” is true, proceed to step 2; if false, the walking rehabilitative device does not move or pause the movement, and the control system continues to determine;
Step 2: the control system controls the rehabilitative device to move at a predetermined speed (Vs). The predetermined speed (Vs) is system default or set by therapist through the operation interface of the control system;
Step 3: the control system acquires the user walking speed from the gait sensing module and calculates the average walking speed (Vn) of the user;
Step 4: the control system compares the predetermined speed (Vs) of the walking rehabilitative device with the average walking speed (Vn) of the user; if {|Vn−Vs|>ϵV} is true, the control system controls the walking rehabilitative device to move at a adaptive speed (Vs1) according to the calculation result of {Vs1=min (Vn, Vu)}; if |Vn−Vs |≤ϵV is true, return to step 2; wherein Vu is set by therapist through the operation interface of the control system; if Vu is not set, Vu is the maximum speed of the walking rehabilitative device, which is preset by the system according to the specification and rotation speed of the motor; wherein (ϵV) is the tolerance value;
Step 5: repeat steps 1 to 4 until the end of training, or forced to stop due to an emergency (such as a user falling).

7. The control method for the rehabilitative device according to claim 3, wherein the high frequency disturbance random speed mode comprising:
Step 1: the control system determines whether the user is located in the user zone through the information from the gait sensing module; if “the user is located in the user zone” is true, proceed to step 2; if false, the walking rehabilitative device does not move or pause the movement, and the control system continues to determine;
Step 2: the control system controls the walking rehabilitative device to high-frequency alternately moves at the predetermined speed (Vs) and a random speed (Vrandom); wherein the occurrence and duration of the predetermined speed (Vs) and the random speed (Vrandom) are randomly controlled by the control system, so that the walking rehabilitative device generates high frequency disturbance random speed movement; the predetermined speed (Vs) is constant, and the random speed (Vrandom) is faster or slower than the predetermined speed (Vs); wherein predetermined speed (Vs) is system default or set by therapist through the operation interface of the control system; wherein the random speed (Vrandom) is system default, set by therapist through the operation interface of the control system, or randomly generated by the control system based on the predetermined speed (Vs);
Step 3: repeat steps 1 and 2 until the end of training, or forced to stop due to an emergency (such as a user falling).

8. The control method for the rehabilitative device according to claim 3, wherein the low frequency perturbation variable speed mode comprising:
Step 1: the control system determines whether the user is located in the user zone through the information from the gait sensing module; if “the user is located in the user zone” is true, proceed to step 2; if false, the walking rehabilitative device does not move or pause the movement, and the control system continues to determine;
Step 2: the control system controls the walking rehabilitative device to low-frequency alternately move at the predetermined speed (Vs) and a variable speed (Vv); wherein the occurrence and duration of the predetermined speed (Vs) and the variable speed (Vv) are randomly controlled by the control system, so that the walking rehabilitative device generates low frequency perturbation variable speed movement; the predetermined speed (Vs) is constant, and the variable speed (Vv) is faster or slower than the predetermined speed (Vs); wherein predetermined speed (Vs) is system default or set by therapist through the operation interface of the control system; wherein the variable speed (Vv) is system default, set by therapist through the operation interface of the control system, or randomly generated by the control system based on the predetermined speed (Vs);
Step 3: repeat steps 1 and 2 until the end of training, or forced to stop due to an emergency (such as a user falling).

9. The control method for the rehabilitative device according to claim 4, wherein the strengthening push/pull mode comprising:
Step 1: the control system determines whether the user is located in the user zone through the information from the gait sensing module; if “the user is located in the user zone” is true, proceed to step 2; if false, the walking rehabilitative device does not move or pause the movement, and the control system continues to determine;
Step 2: the control system acquires the right handle reaction force value (|FR|) and the left handle reaction force value (|FL|) from the multi-axis sensing module;
Step 3: The control system compares the right handle reaction force value (|Fr|) and the left handle reaction force value (|Fl|) with a right preset threshold (Frth) and a left preset threshold (Flth); when {|Fr|>Frth & |Fl|>Flth}, proceed to step 4, otherwise return to step 1; wherein the preset threshold (Frth), (Flth) are system default or set by therapist through the operation interface of the control system;
Step 4: the control system acquires the first human-machine distance dl from the gait sensing module;
Step 5: The control system compares the first human-machine distance (d1), the right handle reaction force value (Fr), and the left handle reaction force value (Fl). If the formula (1) is satisfied, it means the user applies a push force to the walking rehabilitative device. If the formula (2) is satisfied, it means the user applies a pull force to the walking rehabilitative device. Either formula (1) or formula (2) is satisfied, proceed to step 6, indicates that the user; if neither formula (1) nor formula (2) is satisfied, return to step 1; wherein the formula (1) is {(d1 <dznu) & (Fr<0) & (Fl<0)}, the formula (2) is {(d1>dznl) & (Fr>0) & (Fl>0)}, (dznu) is the upper limit of neutral distance; (dznl) is the lower limit of neutral distance; (dznu) and (dznl) are system default;
{(d1<dznu) & (Fr<0) & (Fl<0)} formula (1)
{(d1>dznl) & (Fr>0) & (Fl>0)} formula (2)
Step 6: the rehabilitative device moves according to the pushing direction or the pulling direction at a predetermined speed (Vs);
Step 7: repeat steps 1 to 6 until the end of training, or forced to stop due to an emergency (such as a user falling).

10. The control method for the rehabilitative device according to claim 9, in step 6, the control system also activates the resistance module to increase the movement resistance of the rehabilitative device.

11. The control method for the rehabilitative device according to claim 4, wherein the affected-foot weight-bearing mode comprising:
Step 1: the control system determines whether the user is located in the user zone through the information from the gait sensing module; if “the user is located in the user zone” is true, proceed to step 2; if false, the walking rehabilitative device does not move or pause the movement, and the control system continues to determine;
Step 2: The control system acquires the user's affected side stepping force value (Ta) by the stepping force sensing module;
Step 3: The control system compares the affected side stepping force value (Ta) with a stepping force threshold value (Tth). If {Ta>Tth} is true, proceed to step 4; if false, return to step 1;
wherein the threshold value (Tth) is system default or set by therapist through the operation interface of the control system;
Step 4: The control system detects a duration (Time_Ta) of {Ta>Tth} and compares the duration (Time_Ta) of {Ta>Tth} with a default duration (Time_th). If {Time_Ta<Time_th} is true, return to step 1; if {Time_Ta≥Time_th} is true and the control system acquires the user's healthy side stepping force value (Tc) from the stepping force sensing module, the control system controls the walking rehabilitative device to move a predetermined distance at the predetermined speed (Vs). The predetermined distance is user's stride length, system default or set by therapist through the operation interface of the control system;
Step 5: repeat steps 1 to 4 until the end of training, or forced to stop due to an emergency (such as a user falling).

12. The control method for the rehabilitative device according to claim 4, wherein the affected-hand weight-bearing mode comprising:
Step 1: the control system determines whether the user is located in the user zone through the information from the gait sensing module; if “the user is located in the user zone” is true, proceed to step 2; if false, the walking rehabilitative device does not move or pause the movement, and the control system continues to determine;
Step 2: the control system acquires an affected-hand force vector (Va) and a healthy-hand force vector (Vc) from the multi-axis sensing module;
Step 3: the control system compares the affected-hand force vector (Va) with affected-hand force vector threshold (Vath) and compares the healthy hand force vector (Vc) with a the healthy hand force vector (Vcth); if {|Va|>Vath & |Vc|>Vcth } is true, proceed to step 4; if false, return to step 1; wherein the preset threshold (Vath), (Vcth) are system default or set by therapist through the operation interface of the control system;
Step 4: the control system controls the walking rehabilitative device to move a predetermined distance at the predetermined speed (Vs). The predetermined distance is user's stride length, system default or set by therapist through the operation interface of the control system;
Step 5: repeat steps 1 to 4 until the end of training, or forced to stop due to an emergency (such as a user falling).

13. The control method for the rehabilitative device according to claim 4, wherein the affected-foot-and-hand weight-bearing mode comprising:
Step 1: the control system determines whether the user is located in the user zone through the information from the gait sensing module; if “the user is located in the user zone” is true, proceed to step 2; if false, the walking rehabilitative device does not move or pause the movement, and the control system continues to determine;
Step 2: the control system acquires the user's affected side stepping force value (Ta) from the stepping force sensing module; the control system acquires the affected-hand force vector (Va) and the healthy-hand force vector (Vc) from the multi-axis sensing module;
Step 3: the control system compares the affected side stepping force value (Ta) with a stepping force threshold value (Tth); the control system compares the affected-hand force vector (Va) with affected-hand force vector threshold (Fath) and compares the healthy hand force vector (Vc) with a the healthy hand force vector (Vcth); if {Ta>Tth} and {|Va|>Vath & |Vc|>Vcth} is true, proceed to step 4; if false, return to step 1; wherein the preset threshold (Tth), (Vath), and (Vcth) are system default or set by therapist through the operation interface of the control system;
Step 4: the control system detects a duration (Time_Ta) of {Ta>Tth} and compares the duration (Time_Ta) of {Ta>Tth} with a default duration (Time_th). If {Time_th<Time_th} is true, return to step 1; if {Time_Ta≥Time_th} is true and the control system acquires the user's healthy side stepping force value (Tc) from the stepping force sensing module, the control system controls the walking rehabilitative device to move a predetermined distance at the predetermined speed (Vs). The predetermined distance is user's stride length, system default or set by therapist through the operation interface of the control system;
Step 5: repeat steps 1 to 4 until the end of training, or forced to stop due to an emergency (such as a user falling).

14. The control method for the rehabilitative device according to claim 4, wherein the direction control mode comprising:
Step 1: the control system determines whether the user is located in the user zone through the information from the gait sensing module; if “the user is located in the user zone” is true, proceed to step 2; if false, the walking rehabilitative device does not move or pause the movement, and the control system continues to determine;
Step 2: the control system controls the walking rehabilitative device to move with a predetermined trajectory at the predetermined speed (Vs) or the adaptive speed (Vs1) of claim 6; wherein the predetermined trajectory is generated by trajectory program and including linear movement, left-turn, and right-turn;
Step 3: repeat steps 1 to 3 until the end of training, or forced to stop due to an emergency (such as a user falling).

15. The control method for the rehabilitative device according to claim 4, wherein the direction control mode comprising:
Step 1: the control system determines whether the user is located in the user zone through the information from the gait sensing module; if “the user is located in the user zone” is true, proceed to step 2; if false, the walking rehabilitative device does not move or pause the movement, and the control system continues to determine;
Step 2: the control system acquires the user's right hand force vector (VFr) and the left hand force vector (VFl) from the multi-axis sensing module;
Step 3: the control system compares the user's right hand force vector (VFr) and the left hand force vector (VFl) with a default force vector threshold (VFth); if {(VFl−VFr)>VFth} is true, the control system turns the walking rehabilitative device to the right; if {(VFr−VFl) >VFth} is true, the control system turns the walking rehabilitative device to the left; wherein the default force vector threshold (VFth) is system default or set by therapist through the operation interface of the control system;
Step 4: repeat steps 1 to 3 until the end of training, or forced to stop due to an emergency (such as a user falling).

16. The control method for the rehabilitative device according to claim 4, wherein the direction control mode comprising:
Step 1: the control system determines whether the user is located in the user zone through the information from the gait sensing module; if “the user is located in the user zone” is true, proceed to step 2; if false, the walking rehabilitative device does not move or pause the movement, and the control system continues to determine;
Step 2: the control system acquires the user's right hand force vector (VFr) and the left hand force vector (VFl) from the multi-axis sensing module;
Step 3: the control system compares the user's right hand force vector (VFr) and the left hand force vector (VFl) with a default force vector threshold (VFth); if {(VFl−VFr)>VFth} is true, the control system turns the walking rehabilitative device to the right; if {(VFr−VFl)>VFth} is true, the control system turns the walking rehabilitative device to the left; wherein the default force vector threshold (VFth) is system default or set by therapist through the operation interface of the control system; the control system acquires the distance between the walking rehabilitative device and the obstacle (D0) from obstacle sensing module; the control system compares the distance (Do) with a preset upper threshold (Duth) and a preset lower threshold (Dlth), either {Do>Duth} or {Do<Dlth} is satisfied, the control system controls the walking rehabilitative device to stop and return to step 2, or activate a automatic obstacle avoidance mode or a correction mode to enable the walking rehabilitative device to continue moving and avoid obstacles; or the control system monitors the trajectory of the walking rehabilitative device and compares the trajectory with the predetermined trajectory; If the direction and angle of the movement trajectory deviate significantly from the predetermined trajectory, the control system controls the walking rehabilitative device to stop and return to step 2, or activate a correction mode to correct the direction and angle of the movement to return to the predetermined trajectory;
Step 4: repeat steps 1 to 3 until the end of training, or forced to stop due to an emergency (such as a user falling).

17. The control method for the rehabilitative device according to claim 4, wherein the slope mode comprising:
Step 1: the control system determines whether the user is located in the user zone through the information from the gait sensing module; if “the user is located in the user zone” is true, proceed to step 2; if false, the walking rehabilitative device does not move or pause the movement, and the control system continues to determine;
Step 2: the control system senses that the mobile platform is tilted forward or rearward through tilt sensing module. If the mobile platform is tilted backward, the walking rehabilitative device is going uphill, proceed to step 3; if the mobile platform is tilted forward, the walking rehabilitative device is going downhill, proceed to step 4;
Step 3: the control system acquires the right handle reaction force value (|FR|) and the left handle reaction force value (|FL|) from the multi-axis sensing module; the control system compares the right handle reaction force value (|Fr|) with a right preset threshold (Frth) and compares the left handle reaction force value (|Fl|) with a left preset threshold (Flth); wherein the preset threshold (Frth), (Flth) are system default or set by therapist through the operation interface of the control system; if {Fr≥Fru>0 & Fl≥Flu>0 } is true, indicating that the user applies a pulling force to the walking rehabilitative device, the control system starts the motor to move the walking rehabilitative device at a predetermined speed (Vs);
Step 4: the control system acquires the right handle reaction force value (|Fr|) and the left handle reaction force value (|Fl|) from the multi-axis sensing module; the control system compares the right handle reaction force value (|Fr|) and the left handle reaction force value (|Fl|) with a right preset threshold (Frl) and a left preset threshold (Fll); wherein the preset threshold (Frl), (Fll) are system default or set by therapist through the operation interface of the control system; if {Fr≤Frl<0 & Fl≤Fll<0} is true, indicating that the user applies a pushing force to the walking rehabilitative device, the control system starts the motor to move the walking rehabilitative device at a predetermined speed (Vs);
Step 5: repeat steps 1 to 3 until the end of training, or forced to stop due to an emergency (such as a user falling).
